(19) United States
(12) Patent Application Publication
(10) Pub. No.: US 2021/0038910 A1
(43) Pub. Date: Feb. 11, 2021

(54) SKINCARE SYSTEM PROVIDING AN AROMA LIQUID ATOMIZER UNIT AND A PHOTOTHERAPY UNIT

(71) Applicant: Francis KWOK, Singapore (SG)

(72) Inventor: Francis KWOK, Singapore (SG)

(21) Appl. No.: 16/986,339

(22) Filed: Aug. 6, 2020

Related U.S. Application Data
(60) Provisional application No. 62/883,678, filed on Aug. 7, 2019.

(51) Int. Cl. A61N 5/06; A61M 11/04

(52) U.S. Cl. CPC A61N 5/0616; A61M 11/042; A61N 2005/0652; A61M 2205/051; A61M 2205/3553; A61M 2205/3561; A61M 2202/04; A61N 2005/0663

(57) ABSTRACT

An aroma/skincare therapy and phototherapy device includes: a set of fluid reservoir units; a power source; a main control unit; a low-level light therapy (LLLT) unit; plus a communication unit and/or a user-operating unit. The main control unit is coupled to the power source and the LLLT unit. Each fluid reservoir unit includes a fluid vaporizing element coupled to the main control unit and/or the user-operating unit. The main control unit obtains a set of control signals, and can selectively activate one or more fluid vaporizing elements (providing vapor based facial therapy) and/or the LLLT unit (providing phototherapy) in accordance with the set of control signals. Upper skin surface(s) can be exposed to vapor based facial therapy, and deeper/deepest layers can be exposed to phototherapy. The aroma/skincare therapy and phototherapy device provides synergistic dual aroma/skincare vapor and phototherapy treatment, which can greatly increase skincare therapy flexibility and efficacy.

### CROSS REFERENCE TO RELATED APPLICATION

The present application claims the benefit of U.S. Provisional Patent Application No. 62/883,678, which was filed on 7 Aug. 2019, and which is incorporated herein by reference in its entirety.

### TECHNICAL FIELD

Aspects of the present disclosure relate to a system configured for providing each of aroma/skincare therapy and phototherapy, which in various embodiments includes or is a skincare system having aroma/skincare liquid atomizer unit and a phototherapy unit (e.g., a Low Level Light Therapy (LLLT) unit) that can be activated individually or in combination.

### BACKGROUND

In many countries where relatively high disposable incomes are combined with the desire for an enhanced or idealized appearance fostered by societal pressures, the use of noninvasive therapies for skin disease and skin rejuvenation are on the raise. Our skin is the organ most exposed to light and other pollutions that may cause skin problems. However, it responds well to red and near-infrared (NIR) wavelengths delivered at the correct parameters with therapeutic intent. One statistic shows that in the US alone, the incidence of psoriasis in adults has doubled from 50.8/100,000 in the 1970's to 100.5/100,000 in the 1990's. Other skin diseases are also on the raise due to environmental pollution.

Due to the pursuit of beauty, an ever-increasing number of people use one or more types of skincare treatments to enhance their cosmetic appearance. For instance, skincare solutions or formulations that are applied to (e.g., massaged onto) an individual's skin (e.g., the face and/or neck) can be used in an attempt to enhance the individual's appearance by way of facilitating skin rejuvenation, and/or aid treatment of skin conditions such as acne. Botanical plant extracts such as essential oils are commonly included in the ingredients of skincare solutions, as various essential oils have been found to exhibit anti-bacterial, anti-inflammatory and/or skin-regenerating properties. However, conventional skincare solutions act primarily on the surface or upper layer of the skin, and cannot reliably penetrate into deeper or bottom layers of the skin for aiding skin rejuvenation or the treatment of problematic skin conditions.

Particular types of skincare systems, apparatuses, and devices exist, which can commonly be found, for example, in aesthetic clinics. However, such systems, apparatuses, and devices are often expensive, undesirably complex, and/or large or cumbersome.

A need exists for a skincare system that can affect both surface as well as deeper layers of the skin, and which can be used in a wide variety of settings or locations by individuals outside of a medical or clinical environment (e.g., a beauty clinic), and which can be used without requiring medical or clinical training.

### SUMMARY

To address skin issues or treat skin problems that can be associated with both surface as well as deeper layers of the skin, embodiments in accordance with the present disclosure include or provide a skincare system or apparatus (e.g., a single or unified skincare system or apparatus) that is configurable or configured to act as each of (1) an aromatherapy/aroma-based skincare or skin therapy device, such as an aroma/skincare liquid atomizer; and (2) a phototherapy or light-based skincare or skin therapy device, such as a Low Level Light Therapy (LLLT) device. LLLT is a substantially non-thermal, generally non-thermal, or non-thermal and safe treatment or procedure that involves using low energy light sources such as light emitting diodes (LEDs), e.g., conventional or commercially available LEDs, to excite endogenous chromophores in the skin to elicit photophysical and photochemical effects, processes, or events, such as associated with skin photobiomodulation. Skin photobiomodulation is known to provide skin-rejuvenating effects that stimulate fibroblast proliferation, collagen synthesis, the production of growth factors, and extracellular matrix production by activating cellular mitochondrial respiratory pathways resulting in lifting and tightening lax skin, and the reduction of rhytids. Embodiments in accordance with the present disclosure provide or deliver each of aroma-based skincare or skin therapy techniques and light-based skincare or skin therapy techniques (e.g., in a sequenced or time-separated manner, or simultaneously, depending upon a particular skincare regimen that defines one or more skincare treatment procedures) by way of a single portable, readily-transportable, or compact system or apparatus to address skin issues or treat skin problems at or within different or multiple layers of the skin. By way of embodiments in accordance with the present disclosure, skincare treatment procedures can be simple and straightforward. Moreover, individuals can carry out or perform skincare treatment procedures (e.g., on themselves) in a generally safe, expected safe, or manner, without requiring specialized medical or clinical training.

An object of various embodiments in accordance with the present disclosure is to provide an aroma/skincare therapy and phototherapy/LLLT wellness system, such as a skincare or skin therapy system or unit, which includes each of an aroma/skincare solution atomizer system, subsystem, unit, or device; and a phototherapy system, subsystem, unit, or device, e.g., a low-energy phototherapy or LLLT system, subsystem, unit, or device. For purpose of brevity and simplicity, in the following text, the aroma/skincare solution atomizer system, subsystem, unit, or device may be referred to as an aroma/skincare solution atomizer unit or device; and similarly, the low-energy phototherapy system, subsystem, unit, or device may be referred to as a low-energy phototherapy unit or device. Aromatherapy, in the context of the present disclosure can refer to or be defined as a nature-based or holistic healing treatment that uses natural plant extracts to promote health and well-being. Specifically, in the context of the present disclosure, aromatherapy also includes aroma-based skincare therapy (e.g., by way of dermal exposure to or absorption of plant extracts such as essential oils). Skincare or skin therapy can include facial region or facial skincare or skin therapy, and/or neck region or neck skincare or skin therapy, in a manner readily understood by individuals having ordinary skill in the relevant art. Notwithstanding, embodiments in accordance with the present disclosure can also provide or are applicable to general aromatherapy (olfactory and dermal exposure to or absorption of essential oil), and specific skincare or skin therapy directed to skin or other body regions, such as the hands, wrists, and/or elbows. In the following text, an aroma/skincare liquid atomizer unit that is cooperatively combined, unified, linked, or operationally coordinated with a low-energy phototherapy unit can be referred to as an aroma/skincare liquid atomizer/a low-energy phototherapy unit, or an aroma/skincare liquid atomizer/LLLT unit.

In multiple embodiments, an aroma/skincare liquid atomizer unit and a low-energy phototherapy unit of an aroma/skincare liquid atomizer unit/low-energy phototherapy unit can be selectively or selectably activated individually (e.g., independently, or sequentially) or in combination (e.g., simultaneously, or in a temporally overlapping manner), for instance, under program instruction control by way of user interaction with one or more visual or graphical user interfaces. More particularly, the aroma/skincare liquid atomizer unit is configured for providing aromatherapy/skincare therapy, e.g., at least one of facial region or facial therapy or neck region or neck or other skin therapy, by way of outputting one or more types of aroma/skincare (e.g., aroma/facecare) vapor(s), which can provide sterilizing, anti-inflammatory or moisturizing effects at the skin surface. The low-energy phototherapy unit is configured for providing light therapy, e.g., at least one of facial region or facial light therapy or neck region or neck or other skincare light therapy, by way of outputting light at one or more wavelengths, e.g., bandwidth limited light centered at one or more particular wavelengths, which can provide sterilizing, anti-inflammatory, and/or other skincare effects, such as skin rejuvenating, at the bottom or deepest layer(s) of the skin to achieve deeper care of the skin and improve the skincare effects providable by the system. The system's provision of aroma/skincare therapy as well as low-energy light therapy can synergistically enhance skincare effects achievable by a user.

For instance, in several embodiments an aroma/skincare liquid atomizer unit/low-energy phototherapy unit can provide, deliver, or output low-energy phototherapy to the bottom or deepest layer(s) of the skin by way of the low-energy phototherapy unit, in tandem or concurrent/simultaneous with providing, delivering, or outputting aroma/skincare therapy to the surface or upper layer(s) of the skin by way of the aroma/skincare liquid atomizer unit. The low-energy phototherapy can provide sterilizing, anti-inflammatory, and/or other effects, such as skin rejuvenation, at the bottom or deepest layer(s) of the skin, which can improve, enhance, or optimize one or more aromatherapy skincare effects provided by the aroma/skincare liquid atomizer unit.

With respect to a representative technical solution, in accordance with an aspect of the present disclosure, an aroma/skincare therapy and phototherapy system includes: a housing; a power source; an aroma/skincare unit carried by the housing and coupled to the power source, the aroma/skincare unit including a plurality of aroma/skincare liquid reservoirs, each aroma/skincare liquid reservoir configured for outputting at least one aroma/skincare vapor external to the housing; a low-energy phototherapy unit carried by the housing coupled to the power source and configured for outputting light external to the housing; a control unit carried by the housing and coupled to the power source, the control unit comprising a memory and circuitry configured for activating each of the aroma/skincare unit and the low-energy phototherapy unit in accordance with a set of control signals; and a communication unit and/or a user operation unit configured for providing the set of control signals to the control unit, wherein the control unit is configured for activating, based on the set of control signals, each of: (a) the aroma/skincare unit to output a set of aroma/skincare vapors sourced from: (i) any single aroma/skincare liquid reservoir by itself during a particular time interval; and (ii) two or more aroma/skincare liquid reservoirs in combination during a different time interval, on a selectable or programmable basis; and (b) the low-energy phototherapy unit on a selectable or programmable basis, individually or in combination.

The control unit can be configured for selectively or programmably activating the aroma/skincare unit and the low-energy phototherapy unit relative to each other on a time-sequenced basis or a simultaneous basis in accordance with the set of control signals.

Each aroma/skincare liquid reservoir can include: a container body in which an aroma/skincare fluid can be carried, an aroma/skincare vapor outlet, and an atomizing element coupled to the main control unit and disposed in or adjacent to the container body such that aroma/skincare vapor can exit the aroma/vapor output following activation of the atomizing element; and a memory element storing a numeric code identifying the aroma/skincare liquid reservoir.

A ratio indicated by the set of control signals can determine a working power of an atomization element to be activated, which can be correlated with or correspond to or indicate a rate and/or amount of an aroma/skincare liquid atomized and output as aroma/skincare vapor.

The set of control signals can indicate or include at least one numeric code identifying at least one aroma/skincare liquid reservoir.

The control unit's memory can store a table indicating a correspondence relationship between the numeric code of each aroma/skincare liquid reservoir and a type of aroma/skincare liquid carried by the aroma/liquid reservoir's container body.

The memory element can include a radio frequency identification (RFID) tag, and the aroma/skincare therapy system can include an RFID reader configured for reading the RFID tag of each aroma/skincare liquid reservoir. The RFID reader can be carried by the housing and coupled to the control unit.

The low-energy phototherapy unit can be configurable or configured to perform low-energy phototherapy operations according to wavelength, and wherein a light intensity at one or more wavelengths and a treatment time correspond to the set of control signals. The set of light sources can include a set of light emitting diodes (LEDs).

The set of light sources can include a plurality of light sources configured to output optical signals having wavelengths corresponding to or centered at one or more wavelengths (a) between violet and green, and/or (b) red wavelength portions of the visible optical spectrum

The phototherapy unit can include a set of lenses configured to receive light output by the set of light sources, and output light external to the housing. The set of lenses can be attachable to and removable from a portion of the housing.

The low-energy phototherapy unit can include a printed circuit board (PCB) that is at least partially coated or plated with at least one thermally conductive material; and a subset of light sources can be carried or supported by or embedded on the PCB, wherein at least one aroma/skincare liquid reservoir includes or carries a heat conductive material. The PCB can be disposed in contact with the at least one aroma/skincare liquid reservoir that includes or carries the heat conductive material.

The system can further include a set of magnets carried by the housing, wherein the set of magnets is configured for securely magnetically attaching the housing to a surface of an object to which the set of magnets is magnetically attracted.

The system can further include a user terminal external to the housing, wherein the user terminal includes a personal computing device having a processing unit, a memory storing data and program instruction sets executable by the processing unit, and a set of user interface devices configured for providing a visual or graphical user interface, wherein the user interface is configured to receive user input, and wherein the user terminal is configured for communication with the control unit to establish the set of control signals.

The visual or graphical user interface can provide visual or graphical elements selectable by a user for indicating a user selection of a set of aroma/skincare vapors to be output and/or a type of low-energy phototherapy to be output. The user terminal can communicate with the control unit to establish the set of control signals based on a user input received by way of the visual or graphical user interface.

The user terminal can include one of a smartphone, a tablet computer, and a laptop computer configured for wireless and/or wire-based communication with the communication unit.

In accordance with another aspect of the present disclosure a skincare therapy system or an aroma/skincare liquid atomizer/low-energy phototherapy unit includes: a main control unit; a power source; an aroma/skincare liquid atomizer unit providing at least one aroma/skincare liquid storage device; and a low-energy phototherapy unit providing a set of light sources or illumination devices such as light emitting diodes (LEDs) configured for outputting light or optical signals corresponding to or centered at one or more particular optical wavelengths. In various embodiments, a given aroma/skincare liquid storage device provides a reservoir in which either an aroma fluid or liquid or skincare fluid or liquid can be carried, i.e., the aroma/skincare liquid device stores either an aroma liquid, or a skincare liquid. Notwithstanding, in certain embodiments, an aroma/skincare liquid storage device can carry a combination of an aroma liquid in one reservoir and a skincare liquid in another reservoir. Such a separation of skincare ingredients can minimize or eliminate the need of a preservative or stabilizer, which in practice is often needed to ensure solubility and/or extend product shelf life.

The main control unit is electrically coupled or connected to the power source, the aroma/skincare liquid atomizer unit, and the low-energy phototherapy unit. Each aroma/skincare liquid storage device includes or is formed as a container body and an atomizing element or sheet, wherein the atomizing element is disposed in the container body. Each container body is provided with an aperture, opening, or outlet, e.g., an air/vapor outlet. The atomizing element is electrically coupled or connected to the main control unit. The main control unit includes at least one processing unit or processor (e.g., a data processor) and a memory, and the main control unit is electrically coupled or connected to a communication unit and/or a user operation unit. The main control unit can acquire a set of control signals (e.g., a single control signal, or multiple control signals, depending upon embodiment details) by way of the communication unit and/or the user operation unit. The main control unit can selectively activate the low-energy phototherapy unit according to a control signal, in response to which the low-energy phototherapy unit performs low-energy phototherapy operations including the output of low-energy phototherapy light or optical signals by way of its light source(s). The main control unit can additionally or alternatively activate the atomization element(s) of the aroma/skincare liquid storage device(s) according to a control signal to atomize the aroma/skincare liquid(s) in the aroma/skincare liquid storage device(s).

Each aroma/skincare liquid storage device carries or includes an identification unit, e.g., which is physically couplable, coupled, or connected to its container body. The identification unit stores an identifier (ID) of the aroma/skincare liquid storage device. The main control unit can activate a given aroma/skincare liquid storage device based upon the aroma/skincare liquid storage device's ID, in accordance with or as indicated or specified by a control signal. The atomizing element of the aroma/skincare liquid storage device corresponding to an ID under consideration atomizes the associated aroma/skincare liquid carried by this aroma/skincare liquid storage device. In various embodiments, the aroma/skincare liquid atomizer unit includes a memory for storing a correspondence relationship between the control signal and the ID. In several embodiments, with respect to multiple aroma/skincare liquid storage devices, the memory can store a table indicating a correspondence relationship between the ID of each aroma/skincare liquid storage device and a type of aroma/skincare liquid carried by the container body of the liquid storage device.

Further, the identification unit can include an RFID tag, wherein the aroma/skincare liquid atomizer includes an RFID reader, and the RFID reader is electrically coupled or connected to the main control unit.

Further, the main control unit may activate the atomization element of the aroma/skincare liquid storage device corresponding to the ID specified by a control signal to atomize the corresponding aroma/skincare liquid carried by the aroma/skincare liquid storage device, according to a ratio indicated by the control signal, which determines a working power of the atomization element and which can be correlated with or correspond to or indicate a rate and/or amount of the aroma liquid atomized (e.g., per unit time).

Further, the main control unit can activate the low-energy phototherapy unit according to a control signal to enable the low-energy phototherapy unit to perform low-energy phototherapy operations according to wavelength, where a light intensity at one or more wavelengths (e.g., center wavelengths, where each center wavelength corresponds to bandwidth-limited light) and a treatment time correspond to the control signal.

Further, the light irradiance or energy density can be, but is not necessarily limited to, up to 40 to 60 Joules per square centimeter.

Further, the aroma/skincare liquid atomizer unit includes a power supply interface, and the power supply interface is electrically couplable, coupled, connectable, or connected to the power source and/or the main control unit.

Further, the low-energy phototherapy unit in several embodiments includes a set of light sources, such as a plurality of LEDs, having wavelengths corresponding to or centered at one or more wavelengths (a) between violet and green, and/or (b) red wavelength portions of the visible optical spectrum; and/or a set of optical filter elements configured for outputting wavelengths corresponding to or centered at one or more wavelengths (a) between violet and green, and/or (b) red wavelength portions of the visible optical spectrum. For instance, the set of light sources can be configured for outputting light at center wavelengths of 415 nm, 633 nm, and/or 830 nm. Other wavelengths, e.g., center wavelengths corresponding to bandwidth limited light output by particular LEDs, can be used, depending upon embodiment details. Certain embodiments can include one or more white lights LEDs that can output broadband or “white” light. Optical filter elements can be included to adjust, tailor, or customize one or more output wavelength bands.

Further, the low-energy phototherapy unit can further include a printed circuit board (PCB) that is at least partially coated or plated with at least one thermally conductive material, for instance, an aluminum plate PCB, where at a subset of light sources such as LEDs or each LED is carried or supported by or embedded on the aluminum plate PCB. Each container body can be made of a heat conductive plastic material, and the aluminum plate PCB is disposed in contact with the container body or bodies. The heat generated when one or more LEDs are illuminated is thereby transferred to the aroma/skincare liquid in the container body. With better heat management, the LED light illumination performs better, which in turn enhances the phototherapy skincare treatment.

Further, each container body is provided with a detachable or replaceable cover or container cap. The cover is provided with an air/vapor outlet, and the atomization element and an O-ring are disposed in the cover and covers or seals the air outlet hermetically. The air outlet is carried by or located on a portion of the container body.

In various embodiments, the main control unit, power source, aroma/skincare liquid atomizer unit, and low-energy phototherapy unit are carried or supported by or reside within a common platform or housing. For instance, the housing can include an outer cover including an upper cover, a bottom cover and a middle support member, where the aroma/skincare liquid storage device is mounted on an upper surface of the middle support member, and the power source, the main control unit, and the communication unit can be mounted or positioned on a lower or opposing surface of the middle support member (e.g., the memory can be disposed under the middle support, or embedded in the middle support member). The upper cover is provided with a phototherapy installation position, and the low-energy phototherapy unit is disposed at the phototherapy installation position. The upper cover is matched with the bottom cover to form a cavity, and each aroma/skincare liquid storage device, the middle support member, the power source, the main control unit, and the memory are located in the cavity. The upper cover is provided with a set of vent holes, and a spray generated by atomization of the liquid in a given aroma/skincare liquid storage device can be discharged from the set of vent holes.

Further, the aroma/skincare liquid storage device can be mounted on the middle support member in a detachable manner, and the low-energy phototherapy unit can be mounted on the upper cover in a detachable manner.

Further, a set of magnets can be included, and the set of magnet is disposed on a bottom surface of the bottom cover to enable the housing, and hence the skincare unit (which includes the aroma/skincare liquid atomizer unit and the low-energy phototherapy unit) can be securely or firmly attached or affixed to a material such as a surface of an object to which the magnet is magnetically attracted.

Further, the upper cover can be provided with a plurality of first alignment posts, and the bottom cover can be correspondingly provided with a plurality of second alignment posts. The middle support member can be provided with first alignment pillars, and a plurality of third alignment posts are aligned with the first alignment pillars. The first alignment pillars and the third alignment pillars are provided with electrically conductive magnets, and the magnets of the first alignment pillars are electrically coupled or connected to the low-energy phototherapy unit. A magnet of the third alignment post is electrically coupled or connected to the main control unit. After the first alignment post is engaged with the third alignment post, the magnets in the first alignment post and the third alignment post can mutually contact to achieve electrical coupling or connection between the main control unit and the low-energy phototherapy unit.

Further, at least one lens assembly or lens, e.g., a convex lens, can be disposed above the low-energy phototherapy unit for focusing the light emitted by the low-energy phototherapy unit to concentrate the light at or in a target region or area (e.g., a target spatial or facial region or area).

Further, the lens assembly or lens (e.g., the convex lens) can be installed in a detachable manner to facilitate replacement of the convex lens to adjust the region size or area and/or light intensity output to the region.

In accordance with another aspect of the present disclosure a skincare therapy system includes: a user terminal and an aroma/skincare therapy and phototherapy apparatus, device, or unit, such as an aroma/skincare liquid atomizer/low-energy phototherapy unit provided in accordance with an embodiment of the present disclosure, e.g., as described herein. The user terminal can communicate with the aroma/skincare liquid atomizer/low-energy phototherapy unit through the communication unit, and the user can pass information/data, user input, and/or user selections (e.g., user interface selections) to the user terminal by way of a set of user interface elements, which in various embodiments includes or is a touch screen display device. The user terminal generates and sends a set of controls signal to the aroma/skincare liquid atomizer/low-energy phototherapy unit to selectably or programmably activate the low-energy phototherapy unit and/or the aroma/skincare liquid atomizer unit, e.g., in a manner corresponding to the user input or user selections. The user terminal can include or be, for instance, a portable/personal communication and/or computing device, such as a smartphone/mobile phone, tablet computer, or the like. The user terminal includes a set of user interface elements, such as a display device or screen and one or more types of associated or corresponding user input/output devices (e.g., such a touch screen display that can serve as each of a display device and a user input device). The user terminal is configurable or configured for providing a user interface such as a visual/graphical interface by way of generating visual and/or graphical elements associated with or corresponding to user-selected or user directed operation of the aroma liquid atomizer/low-energy phototherapy unit.

Further, the user terminal stores an executable application program or application (e.g., which includes program instruction sets stored in a memory and executable by way of a processing unit, such as a microcontroller or microprocessor of the user terminal), and when the application program runs, the following processes or methods can be implemented:

Transmitting, according to an input operation of the user, a corresponding control signal to the communication unit of the aroma/skincare liquid atomizer/low-energy phototherapy unit according to an input operation of the user, the main control unit of the aroma/skincare liquid atomizer/low-energy phototherapy unit controlling the delivery or output of low-energy phototherapy and/or aroma/skincare therapy according to the control signal(s), e.g., in a manner set forth herein.

Further, recording corresponding aroma/skincare therapy historical data and/or low-energy phototherapy historical data according to an input operation of the user, and generating a visual or graphical representation of schematic diagram of the aroma/skincare therapy historical data and/or low-energy phototherapy historical data.

Further, where a plurality of aroma/skincare liquid storage devices are provided, each of which has an identification unit storing an ID of the aroma/skincare liquid storage device; the control signal includes: a first control signal for controlling the low-energy phototherapy unit; and a second control signal for controlling the atomization element of at least one of the aroma/skincare liquid storage devices. The first control signal can include the following control parameters: at least one wavelength (e.g., center wavelength) of light emitted by the low-energy phototherapy unit, at least one corresponding light intensity, and a duration of light output. The second control signal can include the following control parameters: the ID of each aroma/skincare liquid storage device to be activated, an atomization ratio of the aroma/skincare liquid of each aroma/skincare liquid storage device, and a corresponding aroma/skincare liquid atomization duration.

Further: determining, according to an input operation of the user, a wavelength value, a light intensity value, and a duration length of the light emitted by the low-energy phototherapy unit to generate a corresponding first control signal and transmitting the same to the aroma/skincare liquid atomizer/low-energy phototherapy unit; and/or determining an ID value, an atomization ratio value, and a duration length of an aroma/skincare liquid storage device to generate a corresponding second control signal and transmitting the same to the aroma/skincare liquid atomizer/low-energy phototherapy unit.

Further, the application program can also implement the following processes or methods:

Receiving an ID to be communicated to aroma/skincare liquid atomizer/low-energy phototherapy unit corresponding to each aroma/skincare liquid storage device, each of the IDs corresponding to a specific aroma/skincare solution; and automatically generating a corresponding aroma/skincare liquid atomization user interface according to the ID and displaying the aroma/skincare liquid atomization user interface on the screen of the user terminal, where the aroma/skincare liquid atomization user interface is provided with a plurality of visual or graphical elements for the user to select, each of the graphical elements corresponding to a control signal that enables the atomizing element of a particular aroma/skincare liquid storage device to be driven.

A corresponding control signal is determined according to a graphical element selected by the user, and the control signal is sent to the aroma/skincare liquid atomizer/low-energy phototherapy unit.

Further, each of the graphical elements can respectively correspond to a specific aroma/skincare liquid mixing ratio, and the mixing ratio is obtained by atomizing the aroma/skincare liquids in two or more aroma/skincare liquid storage devices, in accordance with the control signals corresponding to the graphical elements. The atomization elements in the two or more specific aroma/skincare liquid storage devices are operated in accordance with the mixing ratio.

Further, after the aroma/skincare liquid atomizer/low-energy phototherapy unit receives the control signal(s) corresponding to the graphical element, the main control unit determines, according to the control signal, the atomization element of each aroma/skincare liquid storage device to activate corresponding to the control signal(s). The working power of a given atomization element that will be or which is activated corresponds to or is correlated with the proportion of that atomization element's aroma/skincare liquid which will be or which is atomized by this atomization element during the production of an output aroma/skincare vapor, relative to each other aroma/skincare liquid that will be atomized by way of the activation of any other atomization element during the production of the output aroma/skincare vapor, and the sum of the ratios of the respective aroma/skincare liquids to be atomized is 100%, wherein when the working power of a given atomization element is at a maximum value the proportion of the fluid output corresponding to the atomization element is 100%, and when the working power of the atomization element is zero, the proportion of the fluid output corresponding to that atomization element is 0%.

Further, each of the graphical elements can correspond to a color.

Further, the aroma liquid atomizing user interface can be provided with a label disposed at a position corresponding to a graphical element corresponding to the label, the label indicating a mixing ratio of aroma liquids corresponding to the graphical element. The mixing ratio corresponds to or is associated with physiological effects that are typically expected to or which can be experienced by the user.

Further, the application program can also implement the following processes or methods:

Determining, according to their IDs, whether a plurality of aroma/skincare liquid storage devices store the same aroma/skincare liquid; if so, adjusting the number of graphical elements of the aroma/skincare liquid atomizing user interface, and setting an alternate working condition to alternately use the respective aroma/skincare liquid storage devices storing the same aroma/skincare liquid according to the alternate working conditions. The working conditions are associated with a control signal corresponding to the aroma/skincare liquid.

Further, in some embodiments, when two aroma/skincare liquid atomizer/low-energy phototherapy units are present, after receiving the control signal(s), a first aroma/skincare liquid atomizer unit/low-energy phototherapy unit can forward the control signal to a second aroma/skincare liquid atomizer unit/low-energy phototherapy unit to produce or output two aroma fluids, e.g., concurrently, in tandem, or consecutively. Each aroma/skincare liquid atomizer/low-energy phototherapy unit controls the operation of its own atomizing element(s) according to the control signal(s).

Further, the application program can further provide a low-energy phototherapy user interface, the low-energy phototherapy user interface provided with a low-energy phototherapy parameter selection area, the low-energy phototherapy parameters including: wavelength, light intensity, treatment time, and possibly number of times per week. By way of user input, the user can set the parameters of low-energy phototherapy through the low-energy phototherapy parameter selection area to cause the application program to generate a second control signal corresponding to the low-energy phototherapy parameters set by the user.

Compared with the prior art, a skincare system that includes an aroma/skincare liquid atomizer/low-energy phototherapy unit in accordance with an embodiment of the present disclosure can provide, produce, or output both essential oil vapor spray and low-energy phototherapy light. The aroma/skincare liquid atomizer unit can provide sterilizing, anti-inflammatory or moisturizing effects to the skin surface by way of skincare therapy/aromatherapy, and the low-energy phototherapy unit can provide low-energy phototherapy to a deeper layer of the skin. By way of such low-energy phototherapy, sterilization, anti-inflammatory, skin rejuvenation and/or other effects can be provided to the bottom or deepest layer(s) of the skin, to achieve deeper care of the skin, and greatly improve the skincare effects of the aroma liquid atomizer. Skincare alone through skincare therapy/aromatherapy typically is not or cannot be directly applied to the deeper or deepest layers of the skin, while skincare alone through low-energy phototherapy can make skin unnecessarily or undesirably dry. The typically slow healing process of low-energy phototherapy alone or in isolation (e.g., in the absence of associated aroma/skincare therapy) is also a consideration. Embodiments in accordance with the present disclosure combine aroma/skincare therapy and low-energy phototherapy (e.g., in a time-separated, time sequenced/sequential, or simultaneous manner, such as n a selectable or programmable basis) to achieve synergistic and comprehensive skincare effects on or through all levels of the skin, and avoids the generation of dry skin. The combined therapy or therapies helps accelerate or speed up skin therapy mechanisms and/or effects. The combined therapy or therapies can augment or drive enhanced (e.g., significantly enhanced) skincare therapy mechanisms and/or effects. When combined, each individual skincare therapy can perform more effectively (e.g., significantly more effectively) due to beneficial or synergistic interaction(s) between therapeutic mechanisms and/or effects that would not be initiated, triggered, or achieved with aroma-based skincare therapy alone in the complete absence of phototherapy, and/or phototherapy alone in the complete absence of aroma-based skincare therapy.

### BRIEF DESCRIPTION OF THE DRAWINGS

FIG. 1 is a structural exploded view of an aroma/skincare liquid atomizer provided in accordance with an embodiment of the present disclosure.

FIG. 2 is a schematic structural view of a detachable cover of the aroma/skincare liquid storage device in accordance with an embodiment of the present disclosure.

FIG. 3 is a schematic perspective view of an aroma/skincare therapy and phototherapy in accordance with an embodiment of the present disclosure

FIG. 4 is a first schematic diagram of a skincare liquid atomizing user interface in accordance with an embodiment of the present disclosure.

FIG. 5 is a first schematic diagram of an aroma liquid atomization user interface in accordance with an embodiment of the present disclosure.

FIG. 6 is a second schematic diagram of an aroma liquid atomization user interface in accordance with an embodiment of the present disclosure.

FIG. 7 is a third schematic diagram of an aroma liquid atomization user interface in accordance with an embodiment of the present disclosure.

FIG. 8 is a fourth schematic diagram of an aroma liquid atomizing user interface in accordance with an embodiment of the present disclosure.

FIG. 9 is a schematic diagram of a low-energy phototherapy user interface in accordance with an embodiment of the present disclosure.

### DETAILED DESCRIPTION

Herein, reference to one or more embodiments, e.g., as various embodiments, many embodiments, several embodiments, multiple embodiments, some embodiments, certain embodiments, particular embodiments, specific embodiments, or a number of embodiments, means at least one embodiment, and need not or does not mean or imply all embodiments.

The reference in this specification to any prior publication (or information derived from it), or to any matter which is known, is not, and should not be taken as an acknowledgement or admission or any form of suggestion that prior publication (or information derived from it) or known matter forms part of the common general knowledge in the field of endeavor to which this specification relates.

As used herein, the term “set” corresponds to or is defined as a non-empty finite organization of elements that mathematically exhibits a cardinality of at least 1 (i.e., a set as defined herein can correspond to a unit, singlet or single element set, or a multiple element set), in accordance with known mathematical definitions (for instance in a manner corresponding to that described in An Introduction to Mathematical Reasoning: Numbers, Sets, and Functions, “Chapter 11: Properties of Finite Sets” (e.g., as indicated on p.140) by Peter J. Eccles, Cambridge University Press (1998)). Thus, a set includes at least one element. In general, an element of a set can include or be one or more portions of a system, an apparatus, a device, a structure, an object, a process, a physical parameter, or a value depending upon the type of set under consideration.

The FIGs. included herewith show aspects of non-limiting representative embodiments in accordance with the present disclosure, and features or elements shown in the FIGs. may not be shown to scale or precisely to scale relative to each other. The depiction of a given element or consideration or use of a particular element number in a particular FIG. or a reference thereto in corresponding descriptive material can encompass the same, an equivalent, an analogous, categorically analogous, or similar element or element number identified in another FIG. or descriptive material associated herewith. The presence of “/” in a FIG. or text herein is understood to mean “and/or” unless otherwise indicated.

Particular non-limiting representative embodiments in accordance with the present disclosure will be further described in conjunction with the accompanying FIGs. It should be noted that, without conflict, one or more aspects of the embodiments described below may be arbitrarily combined to form a new embodiment.

Embodiments in accordance with the present disclosure are directed to a wellness system, such as a skincare system that can provide or deliver facial skin therapy, which includes an aroma/skincare liquid atomizer unit/low-energy phototherapy unit providing each of an aroma/skincare liquid atomizer unit configured for providing aromatherapy/skincare therapy, and a phototherapy unit configured for providing phototherapy.

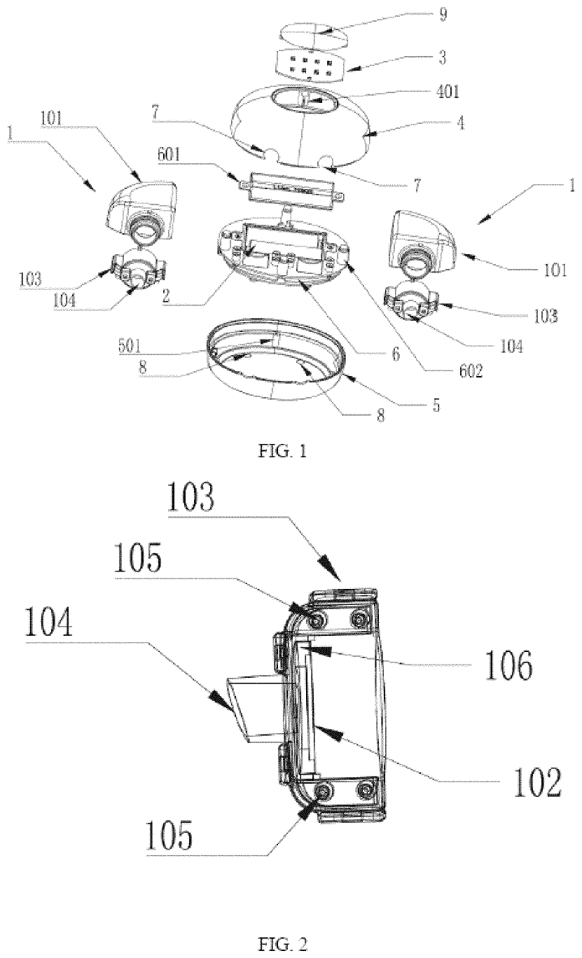

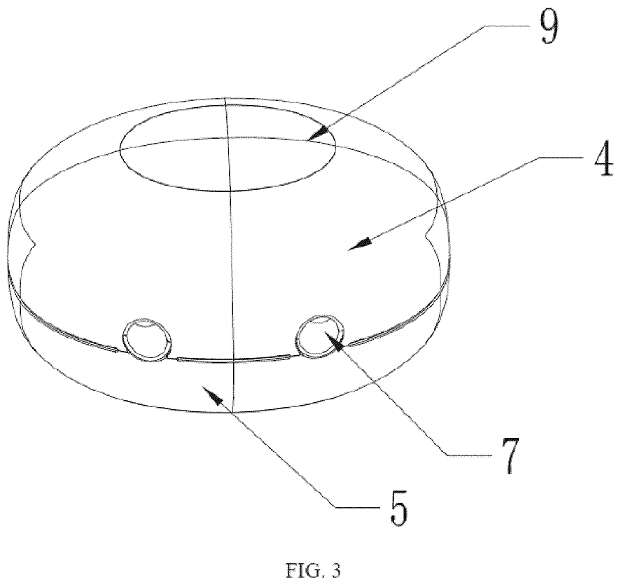

Referring to FIGS. 1 to 3, in various embodiments an aroma/skincare liquid atomizer unit/phototherapy unit includes: an aroma/skincare liquid atomizer unit providing at least one aroma/skincare liquid storage device 1; a power source 2; and a phototherapy unit 3, such as a low-energy phototherapy unit 3, each of which is coupled to a main control unit. The power source 2 can be a battery (e.g., a rechargeable battery), which may be provided for connection to an auxiliary power source (e.g., a mobile power source) and/or a power supply interface of a linear power supply, through which the power source 2 can be charged, or the power supply interface can directly supply power to the aroma/skincare liquid atomizer unit/phototherapy unit. The main control unit can include or be a processing unit or processor such as a microprocessor or microcontroller; or an application specific integrated circuit (ASIC), or Programmable Logic Device (PLD), such as a Field Programmable Gate Array (FPGA) with a corresponding configuration bitstream, or a state machine. The main control unit can also include a memory, e.g., for storing data and one or more program instruction sets executable by the processing unit. In some embodiments, the main control unit includes a clock unit; however, the clock unit can alternatively be separate from the main control unit. In the main control unit, the clock unit is mainly used for the main control unit to control treatment time(s) of the user.

Each aroma/skincare liquid storage device 1 includes a container body 101 and an atomization element or sheet 102. The atomization element 102 can be a piezoelectric transducer or a sprayer. The atomization element 102 is electrically coupled or connected to the main control unit. The atomization element 102 is carried by or disposed on or in a portion of the container body 101. The 101 is provided with an air/vapor passage or outlet, and the atomizing gas can be ejected from the outlet. The aroma/skincare liquid can also be loaded into the container body 101 from the air outlet. A detachable cover 103 is provided, and an air vent 104 is disposed on the cover 103, and the atomization element 102 is disposed in the cover 103 at a position opposite to the air vent 104. The vent 104 may be covered, and the aroma/skincare liquid cannot flow out of the vent 104 due to the blocking action of the (a) o-ring 106 and (b) atomization element 102. When a new aroma/skincare liquid is to be added to the aroma/skincare liquid storage device 1, the cover 103 is detached. The aroma/skincare liquid can be added by way of the air outlet. The o-ring 106 has a dual function of holding the vibrating atomizer and acting as an o-ring between the cover 103 and the container 101 to hermitically seal the fluid within the container body 101. When the cover 103 is mounted over or on the air outlet, the atomization element 102 is in a vertical state or slightly angled, so that particles generated during the atomization process can be prevented from sticking to the atomization element 102 to affect its operation. The air outlet can be disposed at a bottom portion or near bottom of the container body 101 such that the atomization element 102 is located at the bottom portion or near bottom of the container body 101, and the aroma/skincare liquid is always in contact with the atomization element 102 during the process of gradually reducing the level of the aroma/skincare liquid during its atomization. The aperture of the atomization element 102 typically has a pore diameter of 3 micrometers (μm) or larger, e.g., ranging from 3 to 7 μm or larger. When the aperture of the atomization element 102 is 5 μm or larger, the atomization of the skincare liquid produces a vapor stream or vapor mist, which can be applied to the human face. When not used for skincare, for example, for relaxation or tranquilizing effects, an atomization element 102 having a pore diameter of 4 μm or less may be selected, and the atomization chamber produces a mist that acts through the user's olfactory system to the limbic system to provide the desired aromatherapy effect. The cover 103 is provided with an electrical coupling or connection 105 (e.g., a set of coupling/connection points and/or a set of coupling/connection holes) by or through which the atomization element 102 can be electrically connected to the main control unit. In addition, an identification unit can also be carried by or disposed in the cover 103, and the identification unit can be electrically coupled or connected to the main control unit through other electrical coupling or connection structures or elements (e.g., points/holes) in the cover 103.

The aroma/skincare liquid atomizer unit/phototherapy unit further includes a communication unit and/or a user operation unit, and the main control unit operates through the communication unit and/or the user operation unit. The communication unit and/or user operation unit is configured for receiving or acquiring a control signal, such as by way of wireless and/or wireless signal/data communication, e.g., by way of a Bluetooth communication unit, a WiFi communication unit, or a USB interface. The communication unit and/or user operation unit is electrically coupled or connected to the main control unit, and is configured to receive a control signal sent by a user terminal (e.g., a smartphone). The communication unit and/or user operation unit can include a button and/or a touch screen that is disposed on the aroma/skincare liquid atomizer unit/phototherapy unit, which is electrically coupled or connected to the main control unit, and which is configured for receiving user input, e.g., for the user to operate to trigger a corresponding control signal. The main control unit can activate the low-energy phototherapy unit according to the control signal, in order to perform low-energy phototherapy operations, including the generation or output of light or optical signals at particular wavelengths (e.g., the output of bandwidth limited light at one or more center wavelengths). The main control unit can also activate the atomization element 102 of a given aroma/skincare liquid storage device 1 according to the control signal to atomize the aroma/skincare liquid present in the container body 101.

The aroma/skincare liquid atomizer/phototherapy unit can produce, deliver, or output both essential oil spray or vapor and low-energy phototherapy. While one or more aroma/skincare liquid storage devices 1 output aroma/skincare solution(s) providing sterilizing, anti-inflammatory or moisturizing effects to the skin surface by way of upper layer(s) skincare therapy, the low-energy phototherapy unit 3 can provide low-energy light therapy to deeper layer(s) of the skin to achieve deeper care of the skin, e.g., the bottom layer of the skin can receive sterilizing or anti-inflammatory or skin rejuvenation effects by way of phototherapy, thereby improving skincare effects compared to aroma/skin therapy that mainly treats skin problems at the outer layer of the skin via dermal absorption alone. Further to the foregoing, low-energy, low-intensity, or low-level light therapy (LLLT) can be effective or very effective in the treatment of acne problems embattled deep in the inner layers of the skin, and can also have the effect of reducing fine lines, promoting the growth of collagen, and restoring aging skin. However, phototherapy by itself can cause dry skin, and/or its healing duration is longer comparatively, which embodiments in accordance with the present disclosure can avoid or prevent by way of facial spray (topical skincare therapy) in combination with phototherapy. By combining skincare therapy with low-energy light therapy, it is possible to achieve an enhanced care effect on all levels of the skin, and to avoid the problem of dry skin associated with low-energy phototherapy by itself. Of course, when the user only needs to perform one of skincare therapy and low-energy phototherapy, a corresponding control signal can also be sent to the main control unit to control the atomization element 102 or the low-energy phototherapy unit 3 to be activated accordingly.

In various embodiments, the aroma/skincare liquid atomizer/phototherapy unit has a plurality of aroma/skincare liquid storage devices 1, each of which can carry a different aroma/skincare liquid (e.g., which can include an essential oil or other skincare moisturizing substance). Each aroma/skincare liquid storage device 1 is provided with an identification unit, and the identification unit stores an ID corresponding to the aroma/skincare liquid storage device, which can be linked or associated with a particular type of aroma/skincare liquid (e.g., by way of a lookup table). The main control unit can activate the atomization element 102 of an aroma/skincare liquid storage device 1 corresponding to a given ID according to a control signal to atomize the corresponding aroma/skincare liquid. In view of the foregoing, the aroma/skincare liquid atomizer/phototherapy unit includes a memory for storing a correspondence between the control signal and the ID of each aroma/skincare liquid storage device.

It can be noted that in conventional nebulizers for aroma or facial care, there is generally only a single aroma/skincare liquid storage device, and a particular aroma/skincare solution is added to the storage device to achieve a single aromatic or skincare effect. For instance, it is known in the field of aromatherapy that there is synergetic advantage by blending different essential oils for improved therapeutic purposes, and that the “combination is more than the sum of the parts.” However, with a single liquid storage element, impromptu or enhanced-efficacy blending, mixes, or adjustments cannot be achieved. In addition, the skincare solution is usually composed of various plant extracts such as certain essential oils (oil-based) and natural organic substances (water-based), and in most cases, it is necessary to add a solvent, a stabilizer or a preservative to stably mix the various plant extracts together. These solvents, stabilizers or preservatives are inevitably also carried to the user's skin, and have corresponding effects on the user's skin, which may be unwanted, undesired, or unpredictable (e.g., in individuals who may experience certain types of chemical sensitivity reactions). In aroma/skincare liquid atomizer/phototherapy units in accordance with embodiments of the present disclosure, a plurality of aroma/skincare liquid storage devices 1 can be provided, and a plurality of essential oils and moisturizing solution(s) can be stored separately. Generally, when an essential oil(s) and moisturizing solution(s), are stored separately, their stability will be better and the overall combined skincare solution will not easily or rapidly deteriorate. Embodiments in accordance with the present disclosure can thus eliminate the need for the inclusion of solvents, preservatives, and stabilizing substances or chemicals in the aroma/skincare liquids carried by aroma/skincare liquid storage devices 1, while providing among multiple aroma/skincare liquid storage devices 1 multiple individually or separately stored constituent aroma/skincare liquids that have adequate, good, significant or long lasting shelf/usage life, because two or more constituent aroma/skincare liquids can be atomized and output together or combined in vapor form on-demand, e.g., in real time or “on the fly” and on a user responsive/selective/selectable basis, rather than having the different constituents pre-blended.

In addition to the foregoing, an effect of providing a plurality of aroma/skincare liquid storage devices 1 is: by controlling the operating power of the atomization elements 102 of different aroma/skincare liquid storage devices 1 among a plurality of aroma/skincare liquid storage devices, a plurality of different blending/combination ratios of aroma/skincare sprays or vapors, e.g., essential oil sprays or different types of essential oil sprays can be produced, generated, or output, thereby achieving a variety of care effects. For instance, if the user mainly wants to moisturize, they can increase the proportion of the main effect of moisturizing oils, and reduce the proportion of essential oils for the treatment of acne and/or other non-moisturizing effects. When the user wants to treat acne, the proportion of essential oils that are mainly used to treat acne can be increased accordingly, by way of appropriately adjusting (e.g., by way of user selection directed to a visual or graphical user interface) a blending ratio of skincare liquids.

The main control unit can activate the atomization element 102 of an aroma/skincare liquid storage device 1 corresponding to an ID specified or indicated by a control signal to atomize the corresponding aroma/skincare liquid according to the ratio corresponding to the control signal. The main control unit can also adjust the working or operating power or voltage the atomization element 102 that is or which will be activated to atomize its corresponding aroma/skincare liquid during the production of an aroma/skincare vapor. For any given aroma/skincare liquid storage device 1, the proportion of aroma/skincare liquid atomization by its atomization element 102 corresponds to or is correlated with this working or operating power. Total working power, which can be defined as 100% maximum power, can be split or divided between or among multiple aroma/skincare liquid storage devices 1 for which atomization elements 102 are or will be activated to atomize multiple aroma/skincare liquids during the production of the aroma/skincare vapor. For a given aroma/skincare liquid storage device 1, the mixing or blend ratio of its corresponding aroma/skincare liquid relative to the aroma/skincare liquid(s) carried by other aroma/skincare liquid storage devices 1 that are or which will be activated can be adjusted by adjusting the working power, e.g., the relative working power, of the atomization element 102 of the aroma/skincare liquid storage device 1 under consideration. Thus, the operating power of the atomization element 102 is adjustable. For instance, by keeping a constant working voltage of 5V, if the maximum operating current is 160 mA, the operating current in several embodiments can be adjusted, and the adjustment range can be 0 mA to 160 mA. That is to say, the atomization element 102 can operate at any operating current between 0 to 160 mA, and the proportion of the aroma/skincare liquid corresponding to the atomization element 102 also has a maximum value when the atomization element 102 has the maximum power. When working at 160 mA, the atomization ratio of the liquid is at the highest, and as the power of the atomization element 102 decreases, the atomization ratio of the liquid also gradually decreases. In particular, the sum of the operating currents of the atomization elements 102 of the plurality of aroma/skincare liquid storage devices 1 can be set to 160 mA (or higher as the total power consumption is not linear), for example when it is necessary to obtain a mixed or blended or composite spray generated by atomization of two (or more) kinds of aroma/skincare liquids. Individuals having ordinary skill in the art will understand that when the operating current of the atomization element 102 is 90 mA (presumably at 50% or approximately 50% power), the operating current of the 2 atomization elements 102 could be 180 mA instead of 160 mA. The correspondence between the atomization ratio (i.e., the mixing ratio) and the operating current of the atomization element 102 can be previously stored in the memory of the aroma/skincare liquid atomizer/phototherapy unit

In some embodiments, the identification unit is an RFID tag, and the aroma/skincare liquid atomizer includes an RFID reader, and the RFID reader is electrically connected to the main control unit. The encoding of the RFID tag is unique, and the RFID tag is identified by the reader so that the identity of the aroma/skincare liquid storage device 1 can be determined. That is to say, when the user interacts with or directs user input to the user terminal, which can communicate with the aromatic atomizer, or activates a control button or touch screen button of the aroma/skincare liquid atomizer/phototherapy unit, a corresponding control signal can be generated, and the aroma/skincare liquid atomizer/phototherapy unit can determine the mixing ratio of each aroma/skincare liquid to be output in an aroma/skincare vapor blend according to the control signal, and determine the corresponding aroma/skincare liquids to be output by identifying the RFID tags. The aroma/skincare liquid storage device 1 further activates the corresponding atomization element 102 at an operating current corresponding to the corresponding mixing ratio, thereby realizing a mixing ratio of the aroma/skincare spray (e.g., essential oil spray) desired or required by the user.

In several embodiments, the low-energy phototherapy unit 3 includes a plurality of LEDs having wavelengths (e.g., center wavelengths) of 415 nm (blue light), 633 nm (red light) and/or 830 nm (near infrared light). According to the existing research data, blue light is effective for the treatment of inflammatory acne. It absorbs the endogenous porphyrin secreted by Propionibacterium acnes during the growth process by endogenous photodynamic therapy (PDT), which can be referred to as detoxification of P. acnes. The combination of blue light and red light is more effective in treating acne. The main control unit may activate the low-energy phototherapy unit 3 according to the control signal to cause the low-energy phototherapy unit 3 to perform low-energy phototherapy operations, outputting light or optical signals generated by some or all of the LEDs, according to light wavelength, light intensity, the treatment time corresponding to the control signal. Specifically, the light intensity can be adjusted by adjusting the power of the LED, and the light irradiance or energy density in certain embodiments can be up to 40 to 60 Joules per square centimeter, and the light wavelength(s) output by the low-energy phototherapy unit 3 can be adjusted by activating the LED(s) of the corresponding wavelength.

The low-energy phototherapy unit 3 can also use light sources, e.g., LEDs, of additional or other wavelengths or center wavelengths, such as ultraviolet (e.g., near-ultravoilet) light wavelengths and/or infrared (e.g., near-infrared) light wavelengths. In certain embodiments, the low-energy phototherapy unit 3 can include one or more laser diodes. Phototherapy can include ultraviolet therapy, visible light therapy, infrared therapy. Infrared action on the human body mainly improves local blood circulation, promotes swelling and subsidence, relieves pain, reduces muscle tone, relieves tendon and dry exudative lesions. Ultraviolet light acts on the human body, and light energy causes a series of chemical reactions. It has anti-inflammatory, analgesic and anti-caries effects. It is commonly used to treat skin suppurative inflammation and other dermatitis, pain syndrome, rickets or rickets; ultraviolet light with a wavelength range of 310-313 nm. It is referred to as narrow-band UVB (NBUVB), which concentrates on the most active part of ultraviolet light directly affects the affected part of the skin, and filters out harmful ultraviolet rays harmful to the skin. The side effect is small, and it acts on the stratum corneum of the skin. It has been used in major hospitals and clinics treating for psoriasis, vitiligo, chronic eczema, neurodermatitis, atopic dermatitis, palmoplantar pustulosis, pityriasis rosea, alopecia areata, parapsoriasis, chronic ulcers of the skin, sputum-like granulation, and other conditions. Visible light is the light that the human eye can see. The technique of treating diseases with visible light is visible light therapy. It mainly includes red light, blue light, blue violet light and multi-spectral therapy. Red light has an exciting effect; yellow light, green light and red light have the opposite effect; blue purple light can be used to treat nuclear jaundice. A laser generates light amplified by stimulated emission of radiation, which has the characteristics of small divergence angle, good directivity, pure spectrum, good monochromaticity, high energy density, high brightness and good coherence. It has thermal effect, mechanical effect and electromagnetic effect. It can be used for the diagnosis and treatment of many conditions.

In several embodiments, the low-energy phototherapy unit 3 further includes a printed circuit board (PCB) that is at least partially coated or plated with a thermally conductive substance or material, e.g., an aluminum plate PCB, where the LED is thermally coupled or mounted to or embedded on the aluminum plate PCB; and the container body 1 can be made of a thermally conductive material, for instance, a polymer or plastic and/or other type of material. The container body 1 is in contact with the aluminum plate to transfer heat generated by the LEDs to the liquid in the container body 1. Generally, when an LED emits light, heat is generated, and when the heat of the LED is transmitted to the aluminum plate, the temperature of the LED is lowered, and the aluminum plate further transmits heat to the casing of the container body 1. The heat is further transmitted to the liquid in the container body. Generally, the specific heat capacity of the liquid is large, and the heat generated by the LED can be readily absorbed by the liquid. Further, due to the short usage duration of phototherapy (e.g., 5 min to 30 min), the overall temperature of the device is low and ensures the phototherapy functions properly and, at the same time, the stability of the liquids in the containers is not affected.

In various embodiments, a housing is further included, which includes an upper cover 4, a bottom cover 5 and a middle support member 6. The aroma/skincare liquid storage device 1 is detachably mounted on the central support member 6. The power source 2, the main control unit, the communication unit and the memory are disposed below the middle support member 6 or embedded in the middle support member 6. A recess is provided for mounting the battery, the recess seals the battery cover inside the middle support member 6 through a cover plate 601, and the main control unit, the memory and the communication unit are mounted on the central support member 6 in the form of a printed circuit board (PCB). A lower surface of the upper cover 4 is provided with a phototherapy mounting position, and the low-energy phototherapy unit 3 is detachably mounted at the phototherapy mounting position, and the upper cover 4 is matched with the bottom cover 5 to be mounted. A cavity is formed therein, in which the aroma/skincare liquid storage device 1, the middle support member 6, the power source 2, the main control unit and the memory are located. The upper cover 4 is provided with at least one exhaust gas hole 7, such that the aroma mist in the liquid storage device 1 generated spray can be discharged from the exhaust hole 7. All components, such as the liquid storage device 1, the low-energy phototherapy unit 3, the power source 2, and the like are covered by the outer casing to form a unitary structure. The aroma/skincare liquid storage device 1 and the low-energy phototherapy unit 3 are detachably mounted for easy replacement.

In multiple embodiments, the outlet is aligned with the venting opening 7, that is, the air outlet 104 of the cover 103 is aligned with the venting opening 7. When the air outlet and the vent hole 7 are aligned, the distance from the liquid storage device 1 to the outside of the liquid atomizer can be minimized, allowing the vapor to disperse quickly, and facilitate vapor blending more rapidly when two or more liquid reservoir units are operating simultaneously.

Further, the o-ring 106 securely holds the vaporizer element 102 and at the same time serves to hermetically seal the fluid contain within the container 101.

In particular embodiments, since there is a certain gap between the cover 103 and the outer casing, in order to allow the spray to be directly emitted to the outside of the atomizer, the cover 103 is provided with a guiding duct, that is, the air outlet 104 of the cover 103 is the guiding duct. At a pipe mouth, when the length of the guide pipe is too long, some fluid may remain in the pipe, thus affecting the spray effect. To solve this problem, two small center points on the same line can be opened on the guide pipe. The holes, e.g., two small holes, are respectively located on both sides of the pipe, and air flows into the guide pipe from the small holes, thereby forming a flow pressure to push the spray in the pipe from the air outlet 104, so that the excess fluid is not easily retained inside the pipe.

In a number of embodiments, the upper cover 4, and the middle support member 6 are aligned and assembled by a set of alignment posts. The upper cover 4 is typically provided with a minimum of three first alignment posts 401, the middle support member 6 is provided with similar or counterpart second alignment posts 602 for docking the first alignment post 401. A set of magnets is disposed in the alignment post 602 for aligning the first alignment post 401 and the second alignment post 602, and also for transmitting electrical current to the LEDs of the upper cover 4, that is, at the upper cover 4. The first alignment post 401 is provided with a corresponding line connected to the LEDs, and the second alignment post 602 of the middle support 6 is also provided with a corresponding line connected to the power source 2 or the main control unit, and then the power supply 2 provides electrical current which can be transmitted to the first alignment post 401 through the magnets in the second alignment post 602, thereby being transmitted to the LED of the upper cover 4, such that the design does not require wires between the upper cover 4 and the middle support member 6. Such a configuration facilitates quick coupling and decoupling of the low energy phototherapy unit 3, as well as the liquid storage devices or liquid reservoir devices 1. The ease in which multiple liquid storage devices 1, holding different liquids to present cross-contamination, makes this both practical and unique.

In several embodiments, a plurality of additional magnets 8 are further included, and these magnets 8 are disposed on the bottom surface of the bottom cover 5 to enable the aroma/skincare liquid atomizer/phototherapy unit to be firmly fixed on to a surface of an object to which these magnets 8 are magnetically attracted. By providing the magnets 8, the aroma atomizer can be fixed to a surface by magnetic attraction such that it can be relatively firmly attached or fixed to the surface of the object. Such removable and replaceable attachment aid, assist or facilitate the holding of the phototherapy device at an intended, therapeutically practical/useful, or ideal height, distance (e.g., in relation to the focus length of the convex lens 9) and direction to the targeted skin region where the concentration of light is at its highest or most appropriate.

In various embodiments, a convex lens 9 is further disposed above the low-energy phototherapy unit 3 for focusing the light emitted or output by the low-energy phototherapy unit 3 to concentrate the light across a particular spatial region or area. Currently, low-energy phototherapy devices on the market typically have very large LED arrays. The light emitted by these LED arrays covers the entire face of the user. However, many users only need phototherapy for a certain part of their face, and no treatment is needed for other locations. The place where skin is irradiated, but where irradiation is not needed or useful, may exhibit some undesirable or adverse effects, for example, at the eyes. Therefore, embodiments in accordance with the present disclosure concentrate light in a small region or area by way of a set of attachable and de-attachable lenses (e.g., convex lenses) 9, and that the low-energy phototherapy device itself can also be attached easily to any magnetically attachable surface as described above such that the user can perform phototherapy of a specific portion or region of their skin, e.g., a specific portion of their face, by positioning the portion of their skin within the light region or area provided by the convex lens 9.

The light irradiance or energy density can be up to 40 to 60 Joules per square centimeter, e.g., for expected best or optimal effects. Of course, because each person's skin pigment or condition is different, each person's optimum light irradiance or energy density can be different. The set of lenses (e.g., the convex lens) 9 can be mounted in a detachable/reattachable or insertable/removable manner. Therefore, the aroma/skincare liquid atomizer/phototherapy unit can accommodate different replaceable lenses (e.g., convex lenses) 9 for purpose of adjusting the light intensity, irradiance, or energy density and/or the extent of a spatial region or area across which light is output, e.g., by replacement of one convex lens 9 with a different convex lens 9, e.g., having a differences in lens design. Of course, it is also possible to adjust the light intensity, irradiance, or energy density by adjusting the operating power of the LEDs. In addition, it is also possible to modify the size of the concentrated region or area of the light by replacing the lens(es), e.g., convex lenses, 9 of different optical or spherical radii. It should be noted that this region or area refers to the range of light at a position away from the atomizer/phototherapy unit by a predetermined or known distance.

The present invention also provides a wellness or skincare system, e.g., an aroma/skincare therapy/phototherapy system having a user terminal and at least one aroma/skincare liquid atomizer/phototherapy unit as described above, wherein the user terminal can communicate with the aroma/skincare liquid atomizer/phototherapy unit through the communication unit, and the user can direct user input to the user terminal. A control signal is sent to the aroma/skincare liquid atomizer/phototherapy unit by the user terminal to activate the low-energy phototherapy unit and/or the aroma/skincare liquid atomizer unit. The user terminal stores an executable application, and when the application runs, the following processes or methods can be implemented:

Transmitting, according to an input operation of the user, a corresponding set of control signal(s) to the communication unit of the aroma/skincare liquid atomizer/phototherapy unit in response or according to an input operation of the user, the main control unit of the aroma/skincare liquid atomizer/phototherapy unit controlling the low-energy phototherapy unit according to the control signal(s) and/or one or more atomization elements according to the control signal(s).

The user terminal can communicate with the aroma/skincare liquid atomizer/phototherapy unit, and the aroma/skincare liquid atomizer/phototherapy unit can be operated by an application program or app by installing the app on the user terminal.

In several embodiments, the process or method further includes: recording corresponding aromatherapy/skincare history data and/or low-energy phototherapy history data according to an input operation of the user, and generating aromatherapy/skincare therapy historical data schema and/or low-energy phototherapy historical data visual, graphical, or schematic data and/or diagram. The app can also generate a historical data display interface, which is displayed by way of a visual, graphical, or schematic diagram, such as using treatment duration as the ordinate (y-axis) and the date or number of treatments as the abscissa (x-axis) to display the historical therapy data. Aromatherapy/skincare therapy historical data can include mixing ratios, duration, number of times, and the name of the mixed aroma/skincare solutions. Low-energy phototherapy historical data can include wavelength, light intensity, duration, and the like.

In some embodiments, the control signal includes: a first control signal or a first set of control signals for controlling the low-energy phototherapy unit; a second control signal or a second set of control signals for controlling the atomization element(s) 102. The control parameters included in the first control signal are: wavelength, intensity and duration of the light emitted by the low-energy phototherapy unit; and the control parameters included in the second control signal are: ID of the aroma/skincare liquid storage device to be activated; and the atomization ratio and atomization duration for the liquid storage device(s) under consideration.

The process or method can further include: determining, according to an input operation of the user, a wavelength value, a light intensity value, and a duration length of the light emitted by the low-energy phototherapy unit to generate a corresponding first control signal and transmitting the same to the aroma/skincare liquid atomizer/phototherapy unit; and/or determining an ID value, an atomization ratio value, and a duration length of the liquid storage device to generate a corresponding second control signal; and transmitting the same to the aroma/skincare liquid atomizer/phototherapy unit.

The application program may also implement the following processes or methods:

receiving a set of IDs corresponding to aroma/skincare liquid storage devices, e.g., sent by the aroma/skincare liquid atomizer/phototherapy unit, each of the IDs corresponding to a specific liquid within a given liquid storage device; automatically generating a corresponding liquid atomization user interface according to the ID and displaying on the screen of the user terminal, the liquid atomization user interface is provided with a plurality of graphical elements for the user to select, each of the graphical elements corresponding to a control signal that enables the atomization element 102 in a particular aroma/skincare liquid storage device to be driven.

A corresponding control signal is determined according to a graphical element selected by the user, and the control signal is sent to the aroma/skincare liquid atomizer/phototherapy unit. Each of the graphical elements can correspond to a specific mixing ratio, which is obtained by atomizing an aroma/skincare liquid in two or more liquid storage devices. Corresponding control signals enable the atomization elements in two or more particular aroma/skincare liquid storage devices to operate in accordance with the mixing ratio.

After the aroma/skincare atomizer receives the control signal corresponding to the graphical element, the main control unit determines each liquid storage device corresponding to the control signal according to the control signal. The working power of the atomization element 102 thereof corresponds to the proportion of the aroma/skincare liquid associated or linked with the atomization element; and the sum of the ratios of the respective aroma/skincare liquids is 100%; wherein, when the power is at a maximum, the proportion of the liquid corresponding to the atomization element is 100%; and when the working power of the atomization element is zero, the proportion of the liquid corresponding to the atomization element is 0%.

In various embodiments, each of the graphical elements corresponds to a color. The aroma liquid atomizing user interface can further include or provided a label disposed at a position corresponding to a graphical element corresponding to the label, the label indicating a mixture corresponding to the graphical element. The label and the mixture indicate an expected or typical physiological effect of the proportion of the aroma spray on the user.

The application program may also implement the following processes or methods:

Determining, according to the ID, whether a plurality of aroma/skincare liquid storage devices store the same aroma/skincare liquid. If so, adjusting the number of graphical elements of the liquid atomizing user interface, and setting an alternate working condition to alternately use the respective liquid storage devices storing the same aroma/skincare liquid according to the alternate working conditions. The alternate working conditions are associated with a control signal corresponding to the aroma/skincare liquid.

In several embodiments, the aroma/skincare liquid atomizer unit of an aroma/skincare liquid atomizer/phototherapy unit has two or more liquid storage devices 1, e.g., two, three, four, or more liquid storage devices 1.

In some embodiments, multiple aroma/skincare liquid atomizer/phototherapy units can operate (e.g., automatically operate) in a cooperative or coordinated manner relative to each other or together. For instance, after receiving the control signal, a first aroma/skincare liquid atomizer/phototherapy unit can automatically forward the control signal to a second aroma/skincare liquid atomizer/phototherapy unit, such that the first and second aroma/skincare liquid atomizer/phototherapy units each control their own operation according to the control signal.

Further, the application program can further generate or provide a low-energy phototherapy user interface, the low-energy phototherapy user interface providing with a low-energy phototherapy parameter selection area, the low-energy phototherapy parameters including: wavelength, light intensity, treatment time, a number of weeks, and a number of times per week. The user can set the parameters of the low-energy phototherapy through the low-energy phototherapy parameter selection area to cause the application to generate a second control signal corresponding to the low-energy phototherapy parameter set by the user.

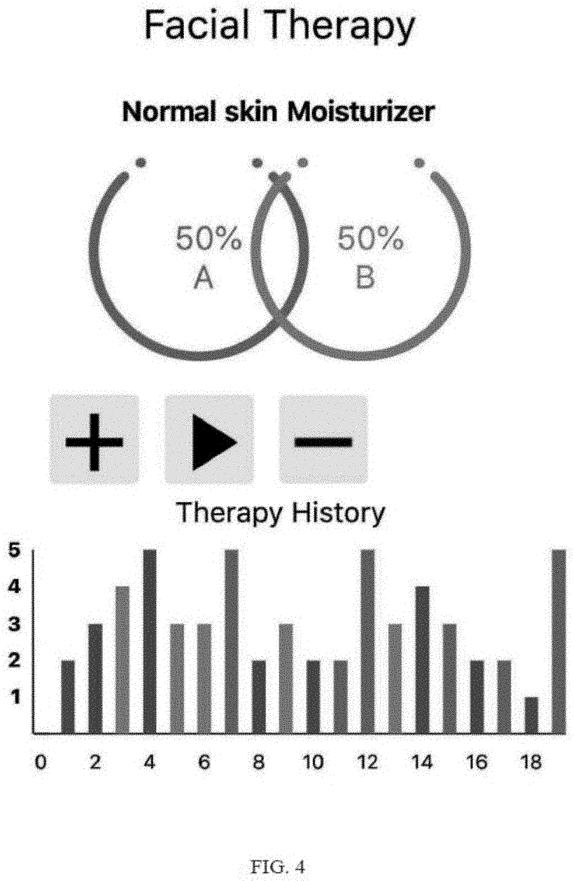

In addition, the skincare liquid atomization interface may also be a user interface as shown in FIG. 4, the user interface corresponding to a skincare liquid atomizer unit having two liquid storage/container device. A percentage adjustment scale is provided to allow the user to adjust the output of storage/container A, with storage/container B self-adjusting to 100%-A %. In the user interface shown in FIG. 4, there is also a schematic diagram of skincare therapy history data, showing historical data of the treatment of the user within a certain period of time (for example, within one month), the historical data may include mixing ratio, duration and the number of treatments. The name of the skincare and moisturizer solution used, and the historical data diagram are shown in FIG. 4. Also included are the duration of the treatment (ordinate), the number of treatments (abscissa) and the mixing ratio (e.g., which can be indicated by colored bars).

It should be noted that the aroma/skincare liquid atomizer/phototherapy unit can be used for both skincare treatment and aromatherapy treatment. When skincare and moisturizer combination is used, the aroma/skincare liquid atomizer unit is used as a skincare or facial sprayer, and when the aromatic essential oil is used, the device functions as an aromatherapy device. The corresponding user interface can be designed separately for the two purposes in the App. When the aroma/skincare liquid atomizer is used for aromatherapy treatment, the aroma liquid atomization user interface is as shown in FIG. 5, in which an arc-shaped visual spectrum or rainbow element such as a rainbow flower is present, and at the left end of the rainbow flower is a graphical element corresponding to red. The right end of the rainbow flower is a graphical element corresponding to purple. From left to right, the color of the graphical element gradually changes, in the order of red orange yellow green blue purple. There are text labels on the edge of the rainbow flower. The text labels can include: “Focus”, “anti-cough”, “anti-flu”, “anti-headache”, “anti-stress”, “sleep”, “sensual” and “ Feminine Comfort”, which indicates the physiological effect of the aromatic vapor composition corresponding to the graphical element corresponding to the label on the user. When the label is a graphical element corresponding to one of the three primary colors (red, green, and blue), it means that only the aromatic vapor of that one aroma corresponding to the color is output, that is, the ratio of the aroma is 100%, and the other aroma stored separately is 0%; and when the label is a graphical element corresponding to the secondary color (for example, orange, yellow, etc.) obtained by mixing two primary colors, it means that the output is obtained by mixing a plurality of aroma solution in proportion to each other.

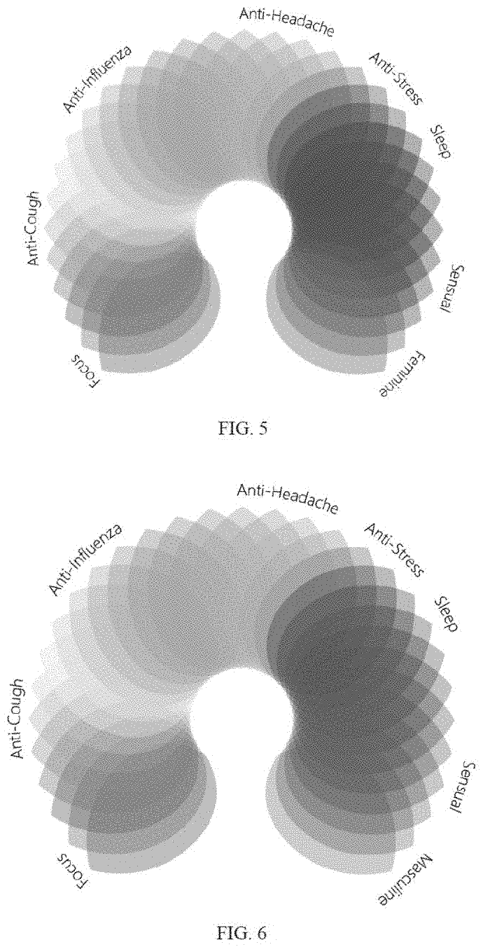

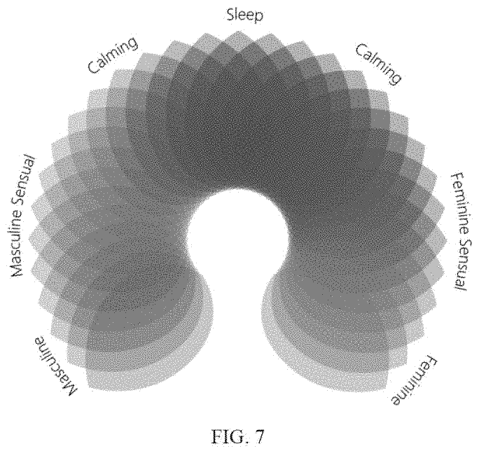

The graphical elements of the rainbow flower that can be selected by the user can be changed based on the ID corresponding to the aroma solution, for example, when there are only two kinds of aromatic solution, the two primary colors correspond to the two aroma solution, such as red and green, then in this interface, only the graphical elements corresponding to the color mixture between red and green can be selected by the user, and the graphical elements between red and green correspond to the specific mixing ratio of the two aroma solutions, and the remaining other colors, such as the graphical elements after green and between blue and purple are not optional. When there are three kinds of aromatic liquids, that is, the IDs of the three aromatic liquid storage devices are presented, the three primary colors correspond to the three aromatic liquids, the interface would then have more color and graphical elements for the user to select. For example, graphical elements from red to blue are optional. The aromatic liquid atomization user interface can also be designed as the interface shown in FIG. 6 or FIG. 7. When the type of the aromatic liquid is different, the physiological effects produced by the aromatic liquid will be different, so the corresponding text label of the graphical element will be correspondingly different. The change, as for the relationship between the text label and the aromatic liquid, may be pre-stored in the memory, and the user terminal may determine the type of aromatic liquid in the aromatic liquid atomizer according to the ID of the identified aromatic liquid storage device, thereby determining the aromatic liquid according to the aroma liquid. A given text label on the user interface can be adjusted accordingly, e.g., in a responsive manner based on one or more IDs. The implementation principle and operation mode of the user interface of FIG. 6 and FIG. 7 are similar or analogous to those of the user interface shown in FIG. 5, as individuals having ordinary skill in the relevant art will comprehend.

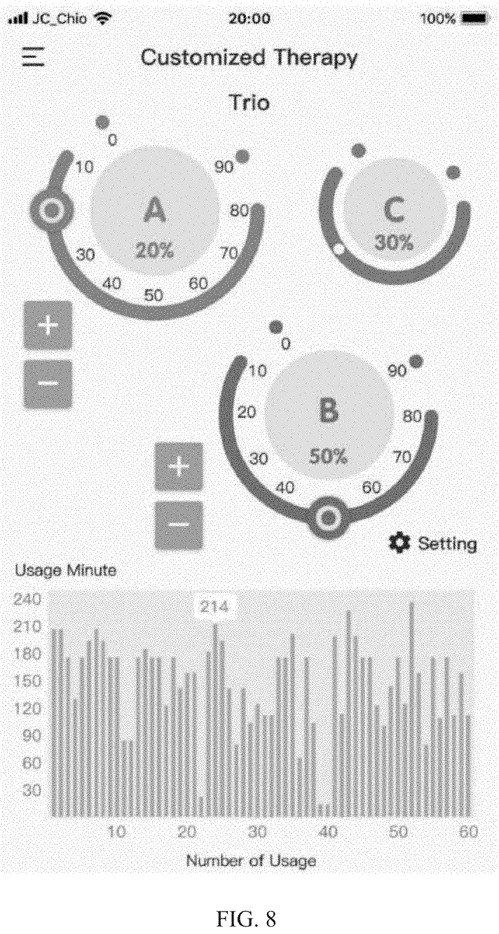

In addition, the aroma liquidation user interface can also be configured as a user interface as shown in FIG. 8, including the IDs of three different aromatic liquid storage devices, each ID corresponding to an aromatic liquid. The three letter labels labeled “A”, “B”, and “C” correspond to three different storages. The app can determine the proportion of the aromatic fluid atomization corresponding to the container (eg. “A”) and the ratio of the other aromatic liquid corresponding to the container “B” according to the user input. The aromatic liquid atomization management app can automatically determine the proportion of the aromatic liquid corresponding to the corresponding container (e.g. “C”), and the proportions of the aromatic liquid corresponding to the aroma/skincare liquid reservoirs or containers “A”, “B” and “C” add up to 100%. The sum of the proportions of the aromatic atomization corresponding to the container “A” and the container “B” is set not to exceed 100% (when it is equal to 100%, the graphic object corresponding to the container “C” will not receive the user input, so that the mixing ratio of the corresponding aromatic steam is 0%). Similarly, the sum of the proportions of the aromatic atomization corresponding to the container “A”, the container “B”, and the container “C” is set at 100%.

In particular, the user interface shown in FIGS. 5 to 7 can provide a plurality of specific mixing ratios of specific aromatic liquids preset in the system, and the user can directly select an intended or desired effect according to the content of the text label. The user interface shown in FIG. 8 can be used for customizing the mixing ratio in response to user input.

Table 1 below provides a representative set of aroma fluid or aroma/skincare (e.g., aroma/facecare) vapor blend percentages that can be provided in response to user selection of graphical elements corresponding to 10%, 20%, 30%, 40%, 50%, 60%, 70%, 80%, and 90% visual labels presented at different “clock face” positions or “clock positions” relative to or around portions of geometric shapes “A” and “B”, referenced in FIG. 8. For geometric shapes “A” and “B,” the aroma/skincare vapor dispensing user interface can provide a standard type of graphical control, e.g., a slider control, that is graphically displaceable about or along portions of the geometric shape in response to user input, thereby enabling aroma/skincare vapor blend ratio selections in response to user input, including blend ratio selections/adjustments in response to user input directed to user-selectable graphical elements located between such “clock positions.”

TABLE 1
Aroma/skincare vapor Blend Percentages Associated with
Visual Labels Displayed at “Clock Positions”
on the Aroma/skincare vapor Dispensing User Interface

| “Clock Position” | | | |
|---|---|---|---|
| on User Interface | A % | B % | C % |
| 10am | 10 | 10-80 | 80-10 |
| 9am | 20 | 10-70 | 70-10 |
| 8am | 30 | 10-60 | 60-10 |
| 7am | 40 | 10-50 | 50-10 |
| 6am | 50 | 10-40 | 40-10 |
| 5am | 60 | 10-30 | 30-10 |
| 4am | 70 | 10-20 | 20-10 |
| 3am | 80 | 10 | 10 |

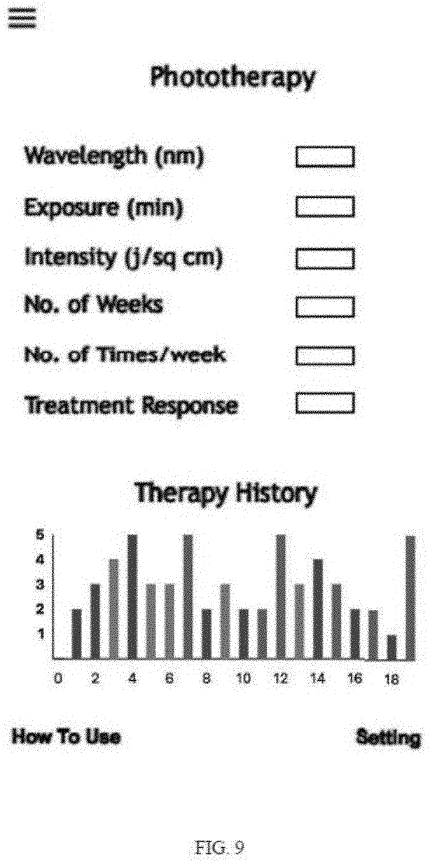

With respect to a low-energy phototherapy interface, the user interface can be as shown in FIG. 9, wherein the wavelength, the treatment time, and the light intensity can be provided by the app with a number of options for the user to select. For example, the wavelength options may include: 415 (blue light), 540 (green light), 633 (red light), 830 (near infrared light) and 415+633 (mixed light); options for treatment time can include: 10, 15, 20, 25, 30 minutes (the treatment time can also be input by the user); and options for light intensity can include: 10, 20, 30, 40, 50, 60, 70, 80 j/sq cm. In addition, the number of weeks of treatment, the number of treatments per week, and the effect of treatment can be received and processed in response to user input. For example, if the user intends to perform low-energy phototherapy for three weeks and three times a week, the user enters “3” for the number of weeks and “3” for the number of times per week, and the system processes such user input accordingly to provide phototherapy in accordance with or responsive to user input. The app will automatically follow the parameters set by the user for the first time (parameters including wavelength, treatment time and light), and can give the user a second and third treatment, etc., without the user having to re-select the parameters. As for the Treatment Response (e.g., intended therapeutic effect), it is not necessary to input the content, and the user can select or input the therapeutic effect according to the light therapy. In the user interface shown in FIG. 9, it also displays phototherapy historical data corresponding to the user, which is presented in the form of a bar graph, where the ordinate is the treatment time, and the abscissa is the number of treatments. The color of each column can be related to phototherapy wavelength, for example, when the phototherapy wavelength is 415 nm, then the color of the strip corresponding to the treatment is blue.

With respect to the foregoing description, embodiments in accordance with the present disclosure enable real-time blending or mixing of two or more types of aroma/skincare liquids, e.g., essential oil carrying liquids. A blended liquid or blended essential oil can be much more therapeutically powerful or useful than a single liquid or essential oil formulation. Additionally or alternatively, embodiments in accordance with the present disclosure enable plant extracts to be carried in separate reservoirs and mixed in real-time or “on-the-fly,” rather than requiring them to be pre-mixed, and thus stabilizers and/or preservatives are not required or can be eliminated in the context of various embodiments in accordance with the present disclosure.

Embodiments in accordance with the present disclosure further enable LLLT to be provided in association or combination with aroma/skincare vapor therapy. LLLT can be focused on particular or targeted areas of the skin (e.g., a specific facial region) by way of a set of lenses. Moreover, embodiments in accordance with the present disclosure can provided multiple user-selectable sets of lenses (e.g., multiple user-selectable convex lenses), or readily user-upgradable sets of lenses.

Embodiments in accordance with the present disclosure provide a compact, portable, personal customizable system, with data recording and data export capabilities (e.g., for review of historical user therapy data, such as by the user themselves, or with a clinician or physician).

In multiple embodiments, a visual or graphical user interface provided by the user terminal (e.g., a smartphone or a tablet/laptop computing device)) employs color mixing or blending in response to user input or user selections, such as mixing of two or three primary colors, to produce a secondary color that is a defined mix ration of the primary colors. Each primary color and each secondary color corresponds to or is representable by a specific numeric code, and hence can correspond to a specific control signal for controlling the aroma/skincare therapy/phototherapy device. A look-up table in a memory stores a plurality of such numeric codes, such that distinct or unique control signals can be recognized and utilized for providing or outputting aroma/skincare therapy and/or phototherapy based on or in a manner correlated with or corresponding to the stored numeric codes.

The above description details non-limiting representative embodiments in accordance with the present disclosure, and the scope of the present disclosure is not limited thereto, and any insubstantial changes and substitutions made by those skilled in the art based on the present description belong to scope of the present disclosure, which is limited only by the following claims.

1. A aroma/skincare therapy and phototherapy system, comprising:
a housing;
a power source;
an aroma/skincare unit carried by the housing and coupled to the power source, the aroma/skincare unit including a plurality of aroma/skincare liquid reservoirs, each aroma/skincare liquid reservoir configured for outputting at least one aroma/skincare vapor external to the housing;
a low-energy phototherapy unit carried by the housing coupled to the power source and configured for outputting light external to the housing;
a control unit carried by the housing and coupled to the power source, the control unit comprising a memory and circuitry configured for activating each of the aroma/skincare unit and the low-energy phototherapy unit in accordance with a set of control signals; and
a communication unit and/or a user operation unit configured for providing the set of control signals to the control unit,
wherein the control unit is configured for activating, based on the set of control signals, each of:
(a) the aroma/skincare unit to output a set of aroma/skincare vapors sourced from:
(i) any single aroma/skincare liquid reservoir by itself during a particular time interval; and
(ii) two or more aroma/skincare liquid reservoirs in combination during a different time interval,
on a selectable or programmable basis; and
(b) the low-energy phototherapy unit
on a selectable or programmable basis.

2. The system of claim 1, wherein the control unit is configured for selectively or programmably activating the aroma/skincare unit and the low-energy phototherapy unit relative to each other on a time-sequenced basis or a simultaneous basis in accordance with the set of control signals.

3. The system of claim 1, wherein each aroma/skincare liquid reservoir comprises:
a container body in which an aroma/skincare fluid can be carried, an aroma/skincare vapor outlet, and an atomizing element coupled to the main control unit and disposed in or adjacent to the container body such that aroma/skincare vapor can exit the aroma/vapor output following activation of the atomizing element; and
a memory element storing a numeric code identifying the aroma/skincare liquid reservoir.

4. The system of claim 3, wherein a ratio indicated by the set of control signals determines a working power of an atomization element to be activated, which can be correlated with or correspond to or indicate a rate and/or amount of an aroma/skincare liquid atomized and output as aroma/skincare vapor.

5. The system of claim 3, wherein the set of control signals indicates or includes at least one numeric code identifying at least one aroma/skincare liquid reservoir.

6. The system of claim 5, wherein the control unit's memory stores a table indicating a correspondence relationship between the numeric code of each aroma/skincare liquid reservoir and a type of aroma/skincare liquid carried by the aroma/liquid reservoir's container body.

7. The system of claim 5, wherein the memory element comprises a radio frequency identification (RFID) tag, and wherein the aroma/skincare therapy system includes an RFID reader configured for reading the RFID tag of each aroma/skincare liquid reservoir.

8. The system of claim 7, wherein the RFID reader is carried by the housing and is coupled to the control unit.

9. The system of claim 1, wherein the low-energy phototherapy unit is configurable or configured to perform low-energy phototherapy operations according to wavelength, and wherein a light intensity at one or more wavelengths and a treatment time correspond to the set of control signals.

10. The system of claim 9, wherein the set of light sources comprises a set of light emitting diodes (LEDs).

11. The system of claim 1, wherein the set of light sources includes a plurality of light sources configured to output optical signals having wavelengths corresponding to or centered at one or more wavelengths (a) between violet and green, and/or (b) red wavelength portions of the visible optical spectrum

12. The system of claim 1, wherein the phototherapy unit includes a set of lenses configured to receive light output by the set of light sources, and output light external to the housing.

13. The system of claim 11, wherein the set of lenses is attachable to and removable from a portion of the housing.

14. The system of claim 1, wherein the low-energy phototherapy unit includes a printed circuit board (PCB) that is at least partially coated or plated with at least one thermally conductive material, wherein a subset of light sources is carried or supported by or embedded on the PCB, and wherein at least one aroma/skincare liquid reservoir comprises a heat conductive material.

15. The system of claim 14, wherein the PCB is disposed in contact with the at least one aroma/skincare liquid reservoir comprising the heat conductive material.

16. The system of claim 1, further comprising a set of magnets carried by the housing, wherein the set of magnets is configured for securely magnetically attaching the housing to a surface of an object to which the set of magnets is magnetically attracted.

17. The system of claim 1, further comprising a user terminal external to the housing, wherein the user terminal comprises a personal computing device having a processing unit, a memory storing data and program instruction sets executable by the processing unit, and a set of user interface devices configured for providing a visual or graphical user interface, wherein the user interface is configured to receive user input, and wherein the user terminal is configured for communication with the control unit to establish the set of control signals.

18. The system of claim 17, wherein the visual or graphical user interface provides visual or graphical elements selectable by a user for indicating a user selection of a set of aroma/skincare vapors to be output and/or a type of low-energy phototherapy to be output.

19. The system of claim 18, wherein the user terminal communicates with the control unit to establish the set of control signals based on a user input received by way of the visual or graphical user interface.

20. The system of claim 17, wherein the user terminal comprises one of a smartphone, a tablet computer, and a laptop computer configured for wireless and/or wire-based communication with the communication unit.
